(19) United States
(12) Patent Application Publication
(10) Pub. No.: US 2021/0402320 A1
(43) Pub. Date: Dec. 30, 2021

(54) CONTINUOUS VACUUM FRACTIONATION SYSTEM FOR SEPARATION OF VARIABLE CANNABIS EXTRACTS

(71) Applicant: Robert Carlton Jones, TAMPA, FL (US)

(72) Inventor: Robert Carlton Jones, TAMPA, FL (US)

(21) Appl. No.: 16/985,719

(22) Filed: Aug. 5, 2020

Related U.S. Application Data
(60) Provisional application No. 63/045,968, filed on Jun. 30, 2020.

(51) Int. Cl. B01D 3/10; C07C 29/76; B01D 8/00; B01D 5/00; B01D 3/14; B01D 3/32

(52) U.S. Cl. CPC B01D 3/106; C07C 29/76; B01D 8/00; B01D 5/0081; B01D 3/143; B01D 3/32; B01D 5/0063

(57) ABSTRACT

A continuous vacuum fractionation system for separation of one or more of the plurality of components present in a raw cannabis extract into at least an overhead fraction, a bottoms fraction, and a side stream fraction, wherein the raw cannabis extract may be prepared by any of a variety of extraction techniques. The system includes an extract supply assembly having a primary feed pump, and one or more continuous fractionation units each including a modular fractionation column. Each column has a re-boiler, a close-coupled overhead condenser, and at least one modular fractionation stage, and further, the columns are operable in either series or parallel configurations. The close-coupled overhead condenser has an oversized impingement plate overlying a condenser inlet to minimize non-vapor components from entering and contacting the condenser. A vacuum assembly is provided to maintain at least the modular fractionation columns under a predetermined vacuum during operation.

### CROSS-REFERENCE TO RELATED APPLICATIONS

This application claims the benefit of U.S. Provisional Patent Application Ser. No. 63/045,968 filed on Jun. 30, 2020, which is incorporated by reference herein in its entirety.

### FIELD OF THE INVENTION

The present invention relates generally to a continuous vacuum fractionation system for the separation of numerous components, compounds, and solvents present in variable amounts in raw cannabis extracts prepared by any of a variety of extraction techniques.

### Background of the Invention

Cannabis has been used for centuries for a variety of purposes including medicinal, food, fibers, such as for rope, cloth, etc., religious ceremonies, and recreationally for its psychoactive properties. At various times throughout history, its use has been restricted, with little large scale enforcement. In the mid-twentieth century, numerous international agreements were reached, leading to large-scale restrictions of the use of cannabis in its various forms due to its psychoactive properties.

Recent legal and market trends have led to an intense demand for cannabidiol, or “CBD”, as well as other components derived from the cannabis plant for various reasons. including a broad range of healing capabilities, as an anti-inflammatory, treatment of sleeplessness, anxiety, etc. However, any cannabis plant with a delta-9-tetrahydrocannabinol, or “THC”, content exceeding 0.3 percent by weight is considered illegal in the United States, making the extraction of CBD from cannabis a highly regulated activity.

In 2018, the United States passed the Farm Bill which established “industrial hemp” as cannabis containing no more than 0.3 percent of THC by dry weight. This classification includes hemp-sourced extracts, cannabinoids, and derivatives in the definition of “industrial hemp”. As a result, a vast market for CBD and related compounds is emerging in the United States, and elsewhere around the world.

The desire to sell CBD and related compounds has led to numerous investigations into various techniques to produce CBD and CBD related products. Simplistically, all these processes contain the same main process steps, though they are not necessarily performed in the same order. A few steps can be performed at different stages in the overall process and these stages are typically chosen for economic and/or process limiting reasons.

The amounts and relative ratios of the desired components and/or compounds present in cannabis differ based on a number of factors such as, the cannabis plant strain, location of harvest, growing season, weather conditions, etc. Cannabis has been found to contain over 550 different components, including at least 113 cannabinoids, including the much sought after cannabidiol (“CBD”) and delta-9-tetrahydrocannabinol (“THC”), as well as over 200 different light, intermediate and heavy terpenes, waxes, chlorophyll, and others. The following are the most common components and/or compounds found in raw cannabis extract: CBD, CBDa, THC, THCa, CBG, CBGa, Waxes, Chlorophyll, Extraction solvent, Terpenes, Linalool, Terpinolene, Phytol, β-Myrcene, Citronellol, Caryphyllene Oxide, α-Pinene, Limonene, β-Caryophyllene, and Humulene.

Regardless of cannabis strain, an extraction step is required to separate the desired components and/or compounds from a cannabis plant matrix using any of a number of possible solvents and extraction techniques. Unfortunately, current extraction practices are far from standardized, and as a result, the raw cannabis extracts being produced vary widely in terms of the amount of desired components and/or compounds present in the raw extract versus the amount of undesirable solvents and/or other contaminants which are commonplace in the various raw cannabis extracts being produced throughout the United States today.

Currently, the process for the separation of individual, relatively pure components from cannabis, such as, for example, CBD, is primarily one of a variety of distillation processes, typically performed in separate batch operations. Each batch distillation operation requires an initial fill of a fixed amount of raw cannabis extract, followed by distillation, shutdown, emptying, cleanout, followed by a refill of the distillation pot with another batch of raw cannabis extract, and the process is repeated. The product of each distillation operation is limited by the total amount of raw cannabis extract processed, the relative concentration of each desired molecular component and/or compound present in the raw cannabis extract feedstock, and the number of individual component collection containers utilized in the batch distillation processes. Distillation only produces one separation product one at a time, based on their differing vaporization temperatures.

Accordingly, there is an established need to resolve one or more of the foregoing problems associated with the separation of the desired components and/or desired compounds from the raw cannabis extracts being produced today by any of a number of various extraction techniques, which vary from very high quality raw cannabis extracts having little to no residual extraction solvents or other contaminants, to very poor quality cannabis extracts replete with residual extraction solvent or solvents and/or any of a variety of undesirable contaminants.

### SUMMARY OF THE INVENTION

The present invention is directed to a continuous vacuum fractionation system for the separation of numerous components, compounds, and solvents present in variable amounts in raw cannabis extracts prepared by any of a variety of extraction techniques.

In a first implementation of the invention, a continuous vacuum fractionation system for separation of one or more of a plurality of components present in a raw cannabis extract, wherein the raw cannabis extract may be prepared by any of a variety of extraction techniques comprises: an extract supply assembly comprising an extract supply and a primary feed pump; a first continuous fractionation unit comprising a first modular fractionation column disposed in fluid communication with the extract supply assembly; the first modular fractionation column having a first re-boiler, a first close-coupled overhead condenser, and at least one first modular fractionation stage; the first close-coupled overhead condenser comprising at least one oversized impingement plate disposed in an at least partially overlying relation to a first condenser inlet from the at least one first modular fractionation stage to minimize non-vapor components from entering and contacting a first condenser therein; a first feed stream comprising an amount of the raw cannabis extract delivered to the at least one first modular fractionation stage of the first continuous modular fractionation column from the extract supply by the primary feed pump; the first continuous modular fractionation column dimensioned and configured to separate the first feed stream into at least a first overhead fraction, a first bottoms fraction, and a first side stream fraction; a second continuous fractionation unit comprising a second modular fractionation column disposed in fluid communication with at least the first continuous modular fractionation unit; the second modular fractionation column having a second re-boiler, a second close-coupled overhead condenser, and at least one second modular fractionation stage; the second close-coupled overhead condenser comprising at least one oversized impingement plate disposed in an at least partially overlying relation to a second condenser inlet from the at least one second modular fractionation stage to minimize non-vapor components from entering and contacting a second condenser therein; a second feed stream delivered to the at least one second modular fractionation stage of the second continuous modular fractionation column, wherein the second feed stream comprises one or more of the raw cannabis extract, the first overhead fraction, the first bottoms fraction, and the first side stream fraction; the second continuous modular fractionation column configured to separate the second feed stream into at least a second overhead fraction, a second bottoms fraction and a second side stream fraction; and, a vacuum assembly disposed in communication with at least the first continuous fractionation unit and the second continuous fractionation unit, the vacuum assembly maintaining at least the first modular fractionation column and the second modular fractionation column under a predetermined vacuum during operation.

In a second aspect, the continuous vacuum fractionation system can include a modular fractionation column comprising a plurality of modular fractionation stages, each of the plurality of modular fractionation stages interconnected in a series arrangement between a re-boiler and a close-coupled overhead condenser, wherein a number of modular fractionation stages is selected based in part upon an assay of the raw cannabis extract being processed.

In one other aspect, the continuous vacuum fractionation system can include a continuous fractionation unit comprising a re-boiler pump disposed to transfer a bottoms fraction from a re-boiler of a modular fractionation column, and a re-boiler airlock disposed between the re-boiler and the re-boiler pump configured to maintain sufficient pressure head at a suction side of the re-boiler pump.

In another aspect, the continuous fractionation system may include a recycle stream comprising all or a portion of a bottoms fraction from a re-boiler of a modular fractionation column which is returned to the re-boiler of the modular fractionation column by a re-boiler pump as may be required to initiate and/or maintain the appropriate temperature in the re-boiler of the modular fractionation column, during startup and/or during operation.

In yet another aspect, the continuous vacuum fractionation system may have a continuous fractionation unit comprising a side stream pump disposed to transfer a side stream fraction from a modular fractionation stage of a modular fractionation column, and a side stream airlock disposed between the modular fractionation stage and the side stream pump configured to maintain sufficient pressure head at a suction side of the side stream pump.

In another aspect, the continuous fractionation system can include the introduction of low pressure steam to one or more modular fractionation stages of a modular fractionation column to increase the efficiency of the overall operation of the continuous fractionation system.

In one further aspect, the continuous vacuum fractionation system can include a vacuum assembly including at least one vacuum pump disposed in communication with one or more continuous fractionation units via a vacuum line, the at least one vacuum pump dimensioned and configured to maintain one or more modular fractionation columns under a predetermined vacuum during operation, wherein the predetermined vacuum is about 0.1 millimeter of mercury to about 100 millimeters of mercury absolute, or the predetermined vacuum is about 0.7 millimeter of mercury absolute, or the predetermined vacuum is about 50 millimeters of mercury absolute.

In another aspect, the continuous vacuum fractionation system may comprise a continuous fractionation unit having a reflux tank wherein an overhead fraction is separated into a liquid reflux which is discharged via a reflux pump, and a reflux vapor which is discharged into a vacuum line.

In still another aspect, the continuous vacuum fractionation system can include a vacuum assembly comprising a cold trap disposed in a vacuum line prior to at least one vacuum pump, the cold trap condenses light extraction contaminants and/or water vapor present in reflux vapor in the vacuum line to minimize the amount of light extraction contaminants which enter the at least one vacuum pump.

In yet one other aspect, the continuous vacuum fractionation system may include a chiller disposed in communication with a cold trap, the chiller configured to maintain the cold trap at a temperature of less than zero degrees Fahrenheit.

In still one further aspect, the continuous vacuum fractionation system can include a condensate discharge dimensioned and configured to transfer condensed light extraction contaminants and/or condensed water vapor from a vacuum line prior to at least one vacuum pump.

In one other aspect, the continuous vacuum fractionation system may include a final product storage assembly including an overhead storage tank, a side stream storage tank, and a bottoms storage tank.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents a diagrammatic representation of one illustrative embodiment of a continuous vacuum fractionation system, in accordance with the present invention;

FIG. 2 presents a diagrammatic representation of the continuous vacuum fractionation system of FIG. 1 including process stream numbers, in accordance with the present invention;

FIGS. 3 through 5 each present a diagrammatic representation of a different portion of the continuous vacuum fractionation system of FIG. 2, in accordance with the present invention;

FIG. 6 presents a diagrammatic representation of one illustrative embodiment of a modular fractionation column, in accordance with the present invention;

FIG. 7 presents a diagrammatic representation of one illustrative embodiment of a close-coupled overhead condenser, in accordance with the present invention; and

FIG. 8 presents a diagrammatic representation of one illustrative embodiment of a modular fractionation stage, in accordance with the present invention.

Like reference numerals refer to like parts throughout the several views of the drawings.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

The present invention is directed to a unique system utilizing one or more modular fractionation columns, each having a uniquely designed close-coupled overhead condenser incorporating an impingement plate, for the separation of a plurality of desirable components and/or compounds present in raw cannabis extract feed stocks of varying quality.

The present system is configured to operate on a continuous basis, in either a series or parallel mode, to produce a plurality of desired separation components and/or compounds from a wide variety of raw cannabis extracts. The system operating parameters may be quickly and easily adjusted to accommodate any of a variety of raw cannabis extracts, ranging from high quality extracts to poor quality extracts, while still producing the desired separation components and/or compounds. As discussed in more detail below, a close-coupled overhead condenser is mounted to a modular fractionation column in a unique manner for the purposes of separating the desired components and/or compounds. The present system employs one or more modular fractionation columns to permit almost infinite operational flexibility, based on the quality of the raw cannabis extract feedstock and/or the desired separation components and/or compounds.

As shown throughout the figures, the present invention is directed toward a continuous vacuum fractionation system for the separation of numerous components, compounds, and extraction solvents and/or extraction contaminants present in variable amounts in raw cannabis extracts prepared by any of a variety of diverse extraction techniques.

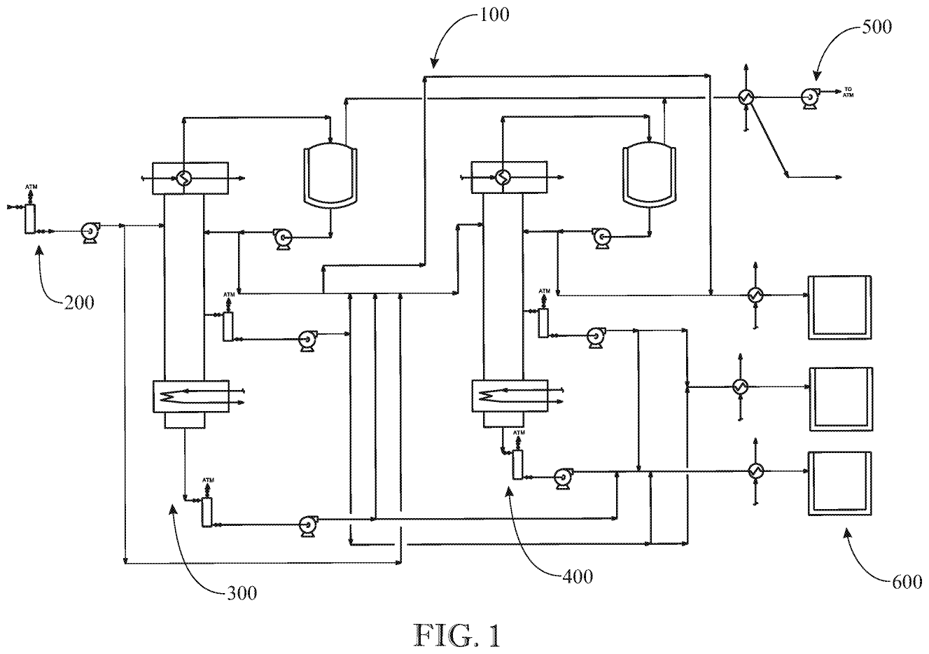

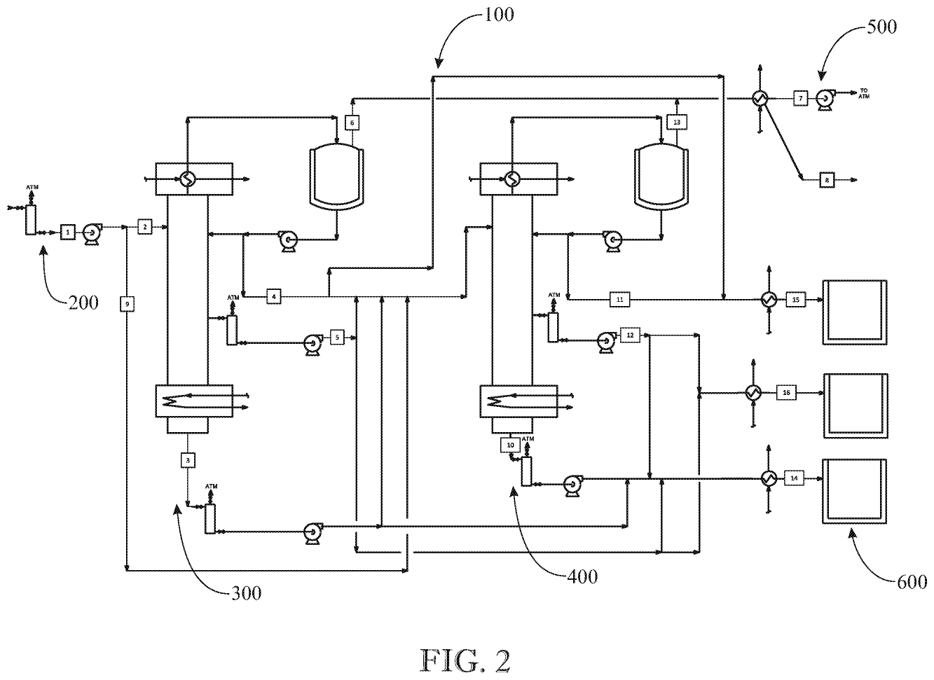

Referring initially to FIGS. 1 and 2, presented therein are diagrammatic representations of one illustrative embodiment of a process flow diagram of a continuous vacuum fractionation system in accordance with the present invention, generally as shown as at 100, wherein FIG. 2 includes process stream numbers 1 through 16, while FIG. 1 does not show process stream numbers. As shown in FIGS. 1 and 2, a continuous vacuum fractionation system 100 includes an extract supply assembly 200 which, as the name implies, is configured to supply amounts of raw cannabis extract to the present system 100 for processing. As before, the present system 100 is configured to effectively and efficiently separate any number of desired separation components and/or compounds from the extraction solvent(s) and/or extraction contaminants present in variable amounts in raw cannabis extracts. More in particular, raw cannabis extracts are prepared using any of a variety of diverse extraction techniques using any of a number of different solvents which result in a very broad spectrum in the “quality” of the raw cannabis extract, as is discussed in more detail below with reference to processing of “good quality” raw cannabis extract versus “poor quality” raw cannabis extract.

With continued reference to FIGS. 1 and 2, a continuous vacuum fractionation system 100 in accordance with at least one embodiment of the present invention comprises a first continuous fractionation unit 300 and a second continuous fractionation unit 400. While illustrated throughout the figures as comprising solely a first continuous fractionation unit 300 and a second continuous fractionation unit 400, a continuous vacuum fractionation system 100 in accordance with the present invention may include additional and/or intermediate fractionation units such as may be warranted depending on the quality of the raw cannabis extract being processed via the present system 100. As will be appreciated by those of skill in the art, and as is clearly shown in the illustrative embodiment of FIGS. 1 and 2, the first continuous fractionation unit 300 and the second fractionation unit 400 may be operated in either a series or a parallel configuration, as well as various combinations of the two. As will be further appreciated, the operational flexibility provided by the present continuous vacuum fractionation system 100 allows the present system 100 to effectively and efficiently separate the numerous desired separation components and/or compounds, from the extraction solvent(s) and/or extraction contaminants present in the various raw cannabis extracts prepared by any of a wide variety of diverse extraction techniques, often with little to no quality control, thus, resulting in a wide range of raw cannabis extract quality.

A continuous vacuum fractionation system 100 in accordance with the present invention further comprises a vacuum assembly 500 which is disposed in communication with at least a first continuous fractionation unit 300 and a second continuous fractionation unit 400, such as is shown by way of example in FIGS. 1 and 2. As also shown in FIGS. 1 and 2, at least one embodiment a continuous vacuum fractionation system 100 in accordance with the present invention further comprises a product storage assembly 600, which is used to at least temporarily store the various fractions produced from a raw cannabis extract after processing through the present system 100.

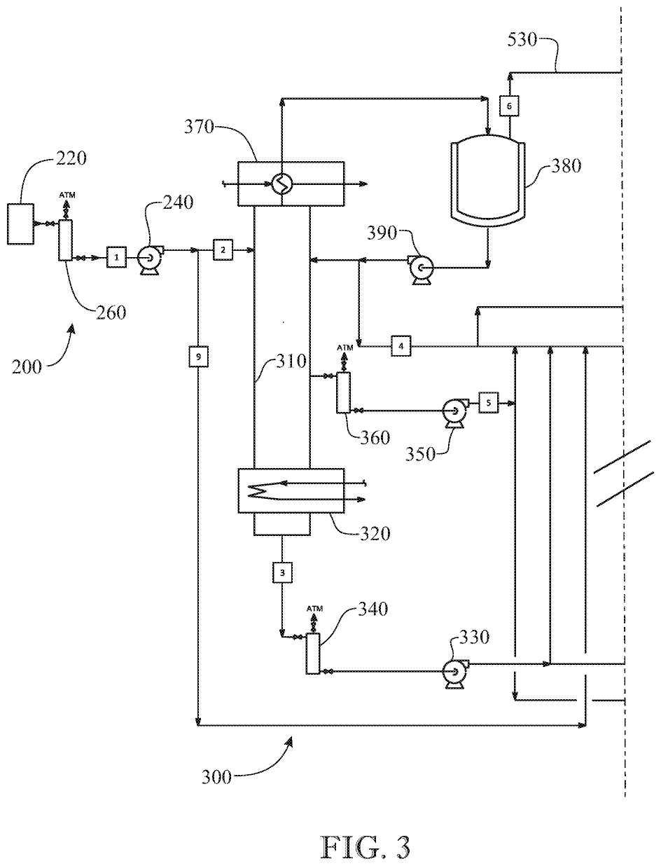

Turning next to FIGS. 3 through 5, different portions of the overall process flow diagram of FIG. 2 are presented in greater detail. Beginning with FIG. 3, and as stated above, a continuous vacuum fractionation system 100 in accordance with at least one embodiment of the present invention includes an extract supply assembly 200. As shown in FIG. 3, an extract supply assembly 200 in at least one embodiment includes an extract supply 220 which may be in the form of an extract storage tank, or a plurality of different extract storage tanks each containing a raw cannabis extract of varying quality. As also shown in FIG. 3, an extract supply assembly 200 comprises a primary feed pump 240 which is utilized to transfer an amount of a raw cannabis extract from an extract supply 220 to at least a first continuous fractionation unit 300. As will be appreciated, at least one embodiment of a continuous vacuum fractionation system 100 in accordance with the present invention an extract supply assembly 200 may comprise a plurality of primary feed pumps 240 in order to transfer amounts of a raw cannabis extract from an extract supply 220 to at least a first continuous fractionation unit 300. As will be further appreciated, a primary feed pump 240 or a plurality of primary feed pumps 240 may be utilized to transfer amounts of a raw cannabis extract from an extract supply 220 to either or both of a first continuous fractionation unit 300 and a second continuous fractionation unit 400.

With continued reference to FIG. 3, in at least one embodiment an extract supply assembly 200 comprises a primary feed airlock 260. More in particular, the present continuous vacuum fractionation system 100 is configured to operate under extreme vacuum which may range from about 0.1 millimeter of mercury (“mmHg”) absolute to about 100 mmHg absolute. In at least one other embodiment, the present system 100 is configured to operate from about 0.5 mmHg absolute to about 75 mmHg absolute. In yet one further embodiment, a continuous vacuum fractionation system 100 is configured to operate under a vacuum of about 0.7 mmHg, and in still another embodiment, the system 100 is configured to operate under a vacuum of about 50 mmHg. As a result of operating under, at times, extreme vacuum conditions, the present continuous vacuum fractionation system 100 incorporates a series of airlocks, such as primary feed airlock 260, in order to assure sufficient head pressure on the suction side of the various transfer pumps utilized throughout the system 100, such as, primary feed pump 240.

Specifically, the continuous vacuum fractionation system 100 of the present invention comprises a plurality of airlocks, such as primary feed airlock 260, which consist of tall narrow tanks having a height which is greater than the height equivalent of the suction head required by a corresponding transfer pump, such as, once again, a primary feed pump 240. Each of the plurality of airlocks, once again, including primary feed airlock 260, includes an inlet valve, an outlet valve, and an atmospheric vent valve, each of which are precisely controlled and actuated in sequence by a computer controller. In operation, initially an inlet valve is opened to allow a liquid to enter, such as, a raw cannabis extract from an extract supply 220, and the airlock tank is allowed to fill with liquid. Based on the level of liquid in the airlock tank, and the suction head required by a corresponding transfer pump, the controller closes the inlet valve, and opens both the outlet valve and the atmospheric vent valve thereby allowing the liquid in the airlock to provide sufficient head pressure to the corresponding transfer pump, once again, such as, a primary feed pump 240. As will be appreciated, by providing a series of airlocks, such as primary feed airlock 260, the present fractionation system 100 is able to operate continuously while remaining under vacuum, even as low as 0.1 mmHg.

The flexibility in the selection of various operating pressures provided by the present continuous vacuum fractionation system 100 facilitates production of relatively pure CBD and/or THC, two of the most sought after separation compounds from raw cannabis extracts today, which may not be attainable by other processes. More in particular, there is a vapor pressure curve cross between CBD and THC which may be advantageously exploited by the present system 100. Specifically, when operated at a pressure of about 0.7 mmHg, below the vapor pressure curve cross, the overhead fraction will be comprised substantially of CBD, while operating at about 50 mmHg, above the vapor pressure curve cross, the overhead fraction will be comprised substantially of THC.

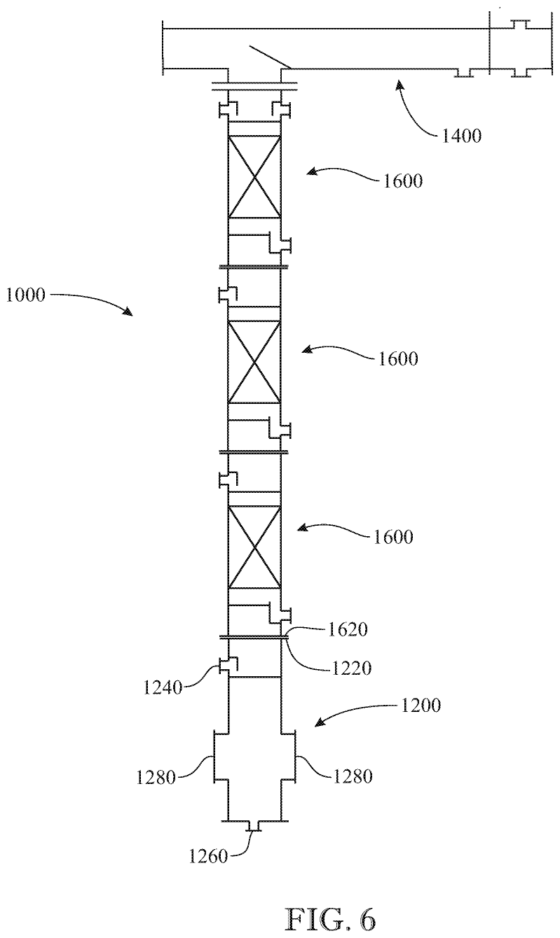

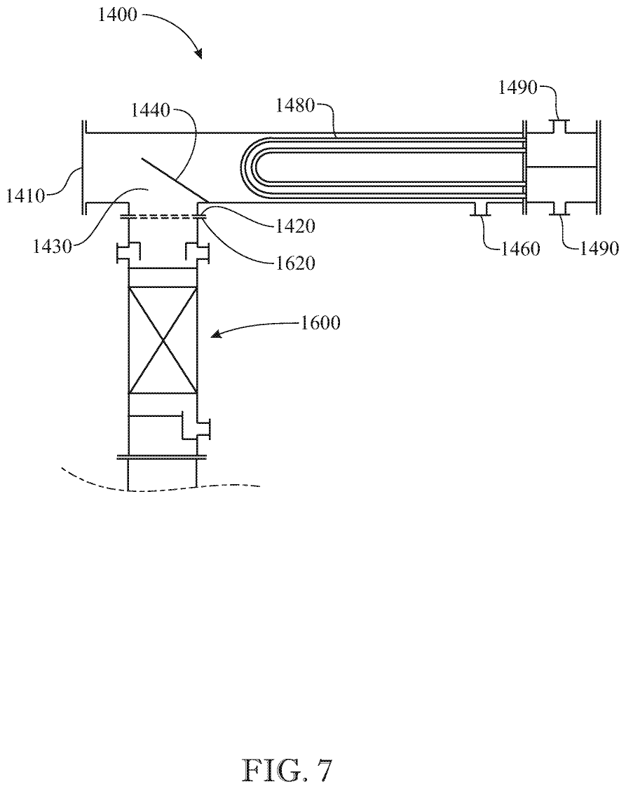

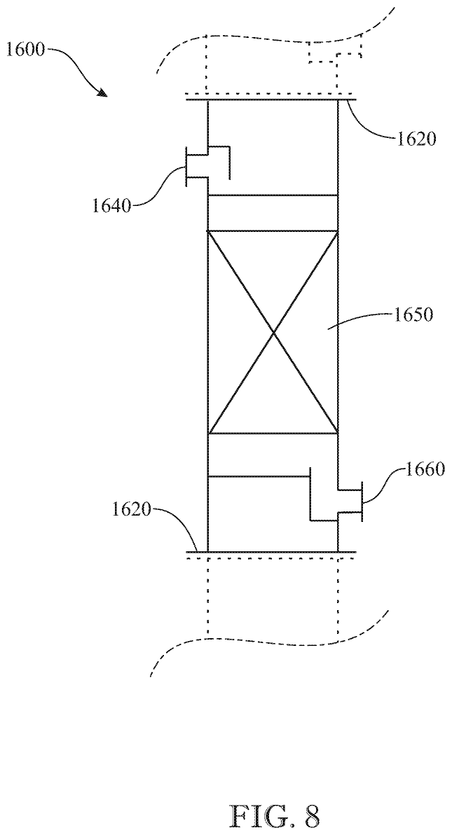

FIG. 3 further presents one illustrative embodiment of a continuous fractionation unit in accordance with the present invention, specifically, a first continuous fractionation unit 300. As may be seen from FIG. 3, the first continuous fractionation unit 300 includes a first modular fractionation column 310. More in particular, the present continuous vacuum fractionation system 100 employs one or more modular fractionation columns each comprising at least one modular fractionation stage such as are discussed in greater detail below with reference to the illustrative embodiments of FIGS. 6 and 8. With reference once again to FIG. 3, a first modular fractionation column 310 comprises a first re-boiler 320 and a first overhead condenser 370. In at least one embodiment, a first modular fractionation column 310 comprises a first close-coupled overhead condenser 370, such as is described in greater detail below with reference to the illustrative embodiment of FIG. 7. As such, a first modular fractionation column 310 in accordance with the present invention is configured to separate a raw cannabis extract into at least a first overhead fraction, a first side stream fraction, and a first bottoms fraction.

With continued reference to FIG. 3, a first continuous fractionation unit 300 further comprises a first re-boiler pump 330 which is disposed in communication with the first re-boiler 320 and is configured to transfer all or a portion of a first bottoms fraction to at least a bottoms storage tank 660, as discussed in greater detail hereinafter. Alternatively, a first re-boiler pump 330 may be utilized to transfer all or a portion of a first bottoms fraction from a first re-boiler 320 to a section of a second continuous fractionation unit 400, such as may be seen in the illustrative embodiment of FIGS. 1 and 2. In at least one embodiment, a first re-boiler pump 330 is utilized to return all or a portion of a first bottoms fraction from a first re-boiler 320 to a section of the first modular fractionation column 310. As also shown in FIG. 3, in at least one embodiment, a first continuous fractionation unit 300 includes a first re-boiler airlock 340. As with the primary feed airlock 260, a first re-boiler airlock 340 is provided to assure sufficient head pressure at all times on the suction side of the first re-boiler pump 330 so as to maintain continuous operation of the present system 100, even while operating under extreme vacuum.

A first continuous fractionation unit 300 in accordance with at least one embodiment of the present invention further comprises a first side stream pump 350 which is disposed in communication with at least one modular fractionation stage of a first modular fractionation column 310. In at least one embodiment, a first side stream pump 350 is configured to transfer all or a portion of a first side stream fraction from a first modular fractionation column 310 to a side stream storage tank 640, as discussed in further detail below. In at least one further embodiment, a first side stream pump 350 is configured to transfer all or a portion of a first side stream fraction from a first modular fractionation column 310 to a section of a second continuous fractionation unit 400, such as is shown in the illustrative embodiment of FIGS. 1 and 2. As may be seen from FIG. 3, in at least one embodiment, a first continuous fractionation unit 300 includes a first side stream airlock 360. As with the primary feed airlock 260 and the first re-boiler airlock 340, a first side stream airlock 360 is provided to assure sufficient head pressure at all times on the suction side of the first side stream pump 350, once again, so as to assure continuous operation of the present system 100 while operating under extreme vacuum.

With continuing reference to the illustrative embodiment of FIG. 3, a first continuous fractionation unit 300 further comprises a first reflux tank 380 disposed in communication with a first close-coupled overhead condenser 370 in accordance with the present invention. More in particular, a first overhead fraction separated from the raw cannabis extract is contacted with a first condenser in the first close-coupled overhead condenser 370 and condensed into a liquid state, which is subsequently transferred to the first reflux tank 380 under vacuum. Specifically, a vacuum line 530 of a vacuum assembly 500 is connected to an upper portion of the first reflux tank 380, as may be seen in FIG. 3. As will be appreciated by those of skill in the art, by virtue of the interconnection between the first reflux tank 380 and the first close-coupled overhead condenser 370, a vacuum is pulled on the entire first continuous fractionation unit 300 by way of the vacuum line 530 of the vacuum assembly 500 being interconnected to the upper portion of the first reflux tank 380.

A first reflux pump 390 is interconnected to the lower portion of the first reflux tank 380, such as is shown by way of example in FIG. 3. In at least one embodiment, the first reflux pump 390 is utilized to return at least a portion of a first liquid reflux fraction from the first reflux tank 380 to the first modular fractionation column 310. In at least one further embodiment, the first reflux pump 390 returns at least a portion of a first liquid reflux fraction to an uppermost modular fractionation stage of the first modular fractionation column 310. The first reflux pump 390 is further utilized to transfer a portion of a first liquid reflux fraction to the second continuous fractionation unit 400 and/or to an overhead storage tank 620, such as is shown throughout the figures.

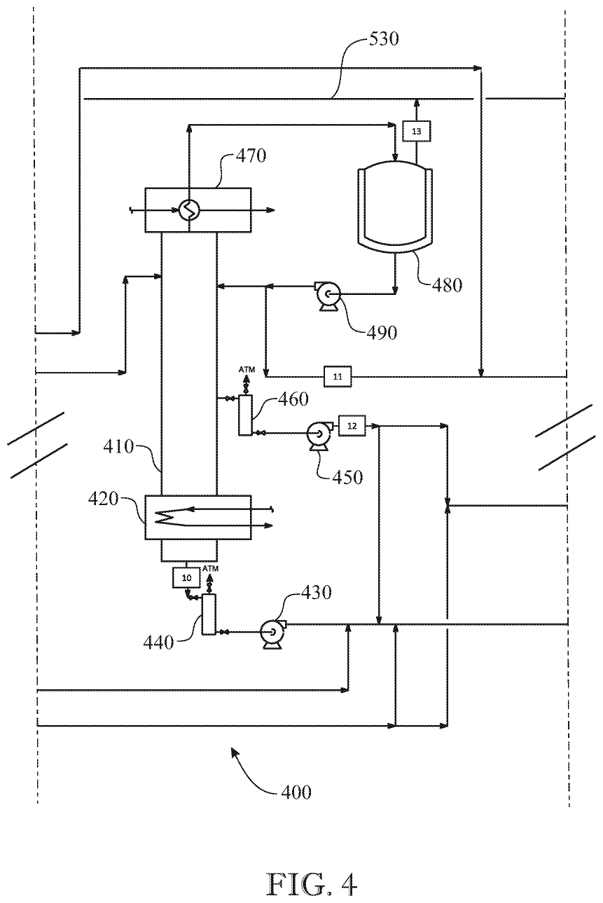

FIG. 4 presents one illustrative embodiment of a second continuous fractionation unit 400. As may be seen from FIG. 4, and similar to the first continuous fractionation unit 300 shown in FIG. 3, the second continuous fractionation unit 400 includes a second modular fractionation column 410. As before, the present continuous vacuum fractionation system 100 employs modular fractionation columns each comprising at least one modular fractionation stage such as are discussed in greater detail below with reference to the illustrative embodiments of FIGS. 6 and 8. With reference once again to FIG. 4, a second modular fractionation column 410 comprises a second re-boiler 420 and a second overhead condenser 470. In at least one embodiment, a second modular fractionation column 410 comprises a second close-coupled overhead condenser 470, once again, such as is described in greater detail below with reference to the illustrative embodiment of FIG. 7. As such, a second modular fractionation column 410 in accordance with the present invention is configured to separate either a raw cannabis extract and/or one or more of a first overhead fraction, a first side stream fraction and/or a first bottoms fraction into at least a second overhead fraction, a second side stream fraction, and a second bottoms fraction.

With continued reference to FIG. 4, a second continuous fractionation unit 400 further comprises a second re-boiler pump 430 which is disposed in communication with the second re-boiler 420, and is configured to transfer a second bottoms fraction to at least a bottoms storage tank 660, as discussed in greater detail hereinafter. Alternatively, a second re-boiler pump 430 may be utilized to transfer all or at least a portion of a second bottoms fraction from a second re-boiler 420 to a section of a second continuous fractionation unit 400, such as may be seen in the illustrative embodiment of FIGS. 1 and 2. In at least one further embodiment, a second re-boiler pump 430 is utilized to return all or a portion of a second bottoms fraction from a second re-boiler 420 to a section of the second modular fractionation column 410. As also shown in FIG. 4, in at least one embodiment, a second continuous fractionation unit 400 includes a second re-boiler airlock 440. As with the first re-boiler airlock 340, a second re-boiler airlock 440 is provided to assure sufficient head pressure at all times on the suction side of the second re-boiler pump 430 so as to maintain continuous operation of the present system 100, even while operating under vacuum.

A second continuous fractionation unit 400 in accordance with at least one embodiment of the present invention also comprises a second side stream pump 450 which is disposed in communication with at least one modular fractionation stage of a second modular fractionation column 410. In at least one embodiment, a second side stream pump 450 is configured to transfer at least a portion of a second side stream fraction from a second modular fractionation column 410 to a side stream storage tank 640, as discussed in further detail below. In at least one further embodiment, a second side stream pump 450 is configured to transfer at least a portion of a second side stream fraction from a second modular fractionation column 410 to a bottoms storage tank 660, depending on the separation components and/or compounds desired in the end product. As may be seen from FIG. 4, in at least one embodiment, a second continuous fractionation unit 400 includes a second side stream airlock 460. As with the first side stream airlock 360, a second side stream airlock 460 is provided to assure sufficient head pressure at all times on the suction side of the second side stream pump 450, once again, so as to assure continuous operation of the present system 100 while operating under extreme vacuum.

With continuing reference to the illustrative embodiment of FIG. 4, a second continuous fractionation unit 400 further comprises a second reflux tank 480 disposed in communication with a second close-coupled overhead condenser 470 in accordance with the present invention. More in particular, a second overhead fraction separated from the raw cannabis extract is contacted with a second condenser in the second close-coupled overhead condenser 470 and condensed into a liquid state, which is subsequently transferred to the second reflux tank 480 under vacuum. As with the first continuous fractionation unit 300, a vacuum line 530 of a vacuum assembly 500 is connected to an upper portion of the second reflux tank 480, as may be seen in FIG. 4. As will be appreciated by those of skill in the art, by virtue of the interconnection between the second reflux tank 480 and the second close couple overhead condenser 470, a vacuum is pulled on the entire second continuous fractionation unit 400 by way of the vacuum line 530 of the vacuum assembly 500 being interconnected to the upper portion of the second reflux tank 480.

A second reflux pump 490 is interconnected to the lower portion of the second reflux tank 480, such as is shown by way of example in FIG. 4. In at least one embodiment, the second reflux pump 490 is utilized to return at least a portion of a second liquid reflux fraction from the second reflux tank 480 to the second modular fractionation column 410. In at least one further embodiment, the second reflux pump 490 returns at least a portion of a second liquid reflux fraction to an uppermost modular fractionation stage of the second modular fractionation column 410. The second reflux pump 490 is further utilized to transfer a portion of a second liquid reflux fraction to the second continuous fractionation unit 400 and/or to an overhead storage tank 620, such as is shown throughout the figures.

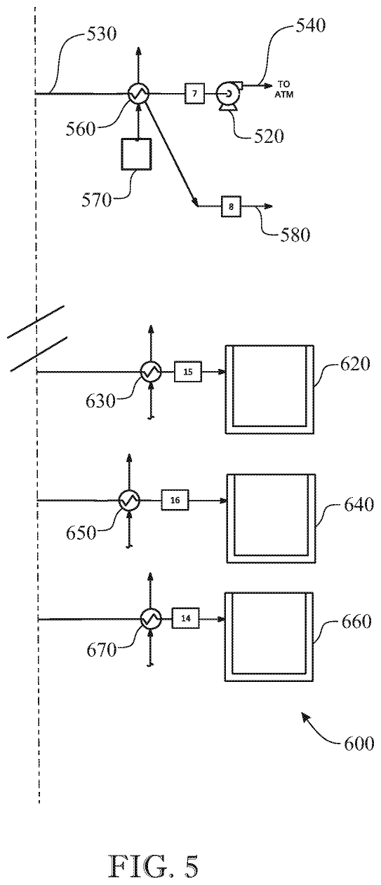

Turning next to the illustrative embodiment of FIG. 5, a continuous vacuum fractionation system 100 in accordance with the present invention further comprises a vacuum assembly 500. As may be seen from FIG. 5, a vacuum assembly 500 includes at least one vacuum pump 520 which is disposed in communication with at least a first continuous fractionation unit 300 and a second continuous fractionation unit 400 via a vacuum line 530. It is appreciated that in at least one embodiment, a vacuum assembly 500 may comprise a plurality of vacuum pumps 520. It is also understood and appreciated that although shown throughout the figures as a single line, a vacuum line 530 in accordance with the present invention may comprise a plurality of interconnected vacuum lines 530 along with appropriate valving arrangements for operation of the present continuous vacuum fractionation system 100.

As before, given the variable nature of the raw cannabis extracts to be processed through the present continuous vacuum fractionation system 100, any of a number of excess extraction solvent(s) and/or extraction contaminants and/or water may be present in the raw cannabis extract, thus, the potential for a number of light end contaminants and/or water becoming entrained in the first reflux vapor and/or the second reflux vapor is considerable. Further, it will be appreciated that such light end contaminants and/or water or water vapor can prove detrimental if not catastrophic to the proper operation of the vacuum pump(s) 520, which is essential for proper operation of the present continuous vacuum fractionation system 100. As such, in accordance with at least one embodiment of the present invention, a vacuum assembly 500 further comprises a cold trap 560 which is disposed in communication with the vacuum line 530 prior to the vacuum pump 520. More in particular, a cold trap 560 is disposed in the vacuum line 530 prior to the vacuum pump 520, wherein the cold trap 560 operates to condenses light extraction contaminants and/or water vapor present in a first reflux vapor and/or a second reflux vapor in present in the vacuum line 530 from the first reflux tank 380 and/or the second reflux tank 480, receptively, to minimize the amount of light extraction contaminants and/or water vapor which enter the vacuum pump 520, thereby minimizing potentially damaging effects thereof.

In at least one embodiment of the present invention, a vacuum assembly 500 further comprises a chiller 570 which circulates a cooling fluid around or through a cold trap 560 such that the cold trap 560 condenses light extraction contaminants and/or water vapor present in a first reflux vapor and/or a second reflux vapor in the vacuum line 530. As such, in at least one embodiment, a chiller 570 is configured to maintain a cold trap 560 at a temperature of less than zero degrees Fahrenheit. In one further embodiment, a chiller 570 is configured to maintain a cold trap 560 at a temperature of about minus ten degrees Fahrenheit. With reference again to FIG. 5, a vacuum assembly 500 in at least one embodiment further comprises a condensate discharge 580 dimensioned and configured to transfer condensed light extraction contaminants and/or condensed water vapor from the vacuum line 530 prior to the inlet of the vacuum pump(s) 520.

As further shown in the illustrative embodiment of FIG. 5, the continuous vacuum fractionation system 100 in accordance with the present invention includes a product storage assembly 600. More in particular, a product storage assembly 600 includes a number of storage tanks such as, by way of example only, an overhead storage tank 620, a side stream storage tank 640, and a bottoms storage tank 660. In one embodiment, one or more heat exchangers may be placed in line prior to one or more of the plurality of storage tanks. As may be seen from FIG. 5, product storage assembly 600 includes an overhead heat exchanger 630 disposed in line prior to overhead storage tank 620, the side stream heat exchanger 650 is disposed in line prior to side stream storage tank 640, and a bottoms heat exchanger 670 is disposed in line prior to bottom storage tank 660. As will be appreciated by those of skill in the art, the plurality of heat exchangers 630, 650, 670 may be utilized to condense vapor which may be present in an overhead fraction, a side stream fraction, and/or a bottoms fraction which are separated from a raw cannabis extract by the present continuous vacuum fractionation system 100. As will be further appreciated by those of skill in the art, at least one of heat exchangers 630, 650, 670, may be utilized to recover heat from one or more of an overhead fraction, a side stream fraction, and/or a bottoms fraction, once again, which have been separated from a raw cannabis extract by the present continuous vacuum fractionation system 100.

Now that a continuous vacuum fractionation system 100 in accordance with the present invention has been fully described, examples of operating temperatures and pressures as well as component flow at each of the process streams identified by process stream numbers 1 through 16 in the illustrative embodiments of FIGS. 2 through 5 are presented.

More in particular, Table I below is exemplary of process conditions throughout a continuous vacuum fractionation system 100 in accordance with the present invention utilizing a “good” quality raw cannabis extract as a feedstock to the system 100. In general, a “good” quality raw cannabis extract is generally defined as a raw cannabis extract containing the following in addition to the primary separation components and/or compounds desired: little to no amount of the extraction solvent used; little to no waxes and/or paraffins; little to no light terpenes; some intermediate and heavy terpenes; and, little to no other undefined, non-essential oil components.

When processing a “good” quality raw cannabis extract through the present continuous vacuum fractionation system 100, a first continuous fraction unit 300 and a second continuous fraction unit 400 may be operated in a parallel configuration. As a result, and as may be seen in from Table I and Table II below, the throughput through the system 100, as a whole, may be doubled. For example, the total raw cannabis extract feed to the system 100 in the example in Table I below is 60 pounds per hour (lb/hr) with 30 lb/hr directed to the first continuous fractionation unit 300 and 30 lb/hr directed to the second continuous fractionation unit 400, while in the example in Table II, a total of 30 lb/hr is directed to the first continuous fractionation unit 300, and only portion thereof are actually transferred as feedstock to the second continuous fractionation unit 400. As may be further seen from Table I, the compositions of the feed stream, the bottoms fractions, the side stream fractions, and the overhead fractions of the first and second continuous fractionation units 300, 400 are identical.

TABLE I
Exemplary Processing of a “Good” Quality Raw Cannabis Extract
Two Continuous Fractionation Units Operating in Parallel

| | Steam No. | | | | | | | |
|---|---|---|---|---|---|---|---|---|
| | 1 | 2 | 3 | 4 | 5 | 6 | 7 | 8 |
| | Comp. Flow (lb/hr) | | | | | | | |
| | Ttl Raw Ext | Frac Col #1 | Frac Col #1 | Frac Col #1 | Frac #1 | Frac Col #1 | System Vac | Cold Trap |
| | Feed to System | Feed | Btms | Ovhds | Intermed | Vac Strm | Pump Outlet | Condensate |
| CBD | 43.8 | 21.9 | 0.0 | 21.2 | 0.7 | 0.0 | 0.0 | 0.0 |
| THC | 6.6 | 3.3 | 0.2 | 0.0 | 3.1 | 0.0 | 0.0 | 0.0 |
| Lt. Terpenes | 0.0 | 0.0 | 0.0 | 0.0 | 0.0 | 0.0 | 0.0 | 0.0 |
| Inter. Terpenes | 5.1 | 2.6 | 0.1 | 0.0 | 2.5 | 0.0 | 0.0 | 0.0 |
| Hvy. Terpenes | 4.5 | 2.3 | 2.2 | 0.0 | 0.1 | 0.0 | 0.0 | 0.0 |
| Solvent | 0.0 | 0.0 | 0.0 | 0.0 | 0.0 | 0.0 | 0.0 | 0.0 |
| Heavy Oils | 0.0 | 0.0 | 0.0 | 0.0 | 0.0 | 0.0 | 0.0 | 0.0 |
| Chlorophyll | 0.0 | 0.0 | 0.0 | 0.0 | 0.0 | 0.0 | 0.0 | 0.0 |
| Waxes/Paraffins | 0.0 | 0.0 | 0.0 | 0.0 | 0.0 | 0.0 | 0.0 | 0.0 |
| Total (lb/hr) | 60.0 | 30.0 | 2.5 | 21.3 | 6.3 | 0.0 | 0.0 | 0.0 |
| Temp (° F.) | 125.0 | 125.0 | 420.0-470.0 | 150.0 | 410.0-460.0 | 150.0 | 85.0 | 25.0 |
| Pressure (psig) | 1.0 | 15.0 | −13.0 | 30.0 | 30.0 | −14.688 | −14.688 | −14.688 |
| | Stream No. | | | | | | | |
| | 9 | 10 | 11 | 12 | 13 | 14 | 15 | 16 |
| | Comp. Flow (lb/hr) | | | | | | | |
| | Frac | Frac | Frac | Frac | Frac | Combined | Combined | Combined |
| | Col #2 | Col #2 | Col #2 | Col #2 | Col #2 | Column | Column | Column |
| | Feed | Btms | Ovhds | Intermed | Vac Strm | Btms | Ovhds | Intermeds |
| CBD | 21.9 | 0.0 | 21.2 | 0.7 | 0.0 | 0.0 | 42.5 | 1.3 |
| THC | 3.3 | 0.2 | 0.0 | 3.1 | 0.0 | 0.4 | 0.1 | 6.1 |
| Lt. Terpenes | 0.0 | 0.0 | 0.0 | 0.0 | 0.0 | 0.0 | 0.0 | 0.0 |
| Inter. Terpenes | 2.6 | 0.1 | 0.0 | 2.5 | 0.0 | 0.2 | 0.0 | 4.9 |
| Hvy. Terpenes | 2.3 | 2.2 | 0.0 | 0.1 | 0.0 | 4.4 | 0.0 | 0.1 |
| Solvent | 0.0 | 0.0 | 0.0 | 0.0 | 0.0 | 0.0 | 0.0 | 0.0 |
| Heavy Oils | 0.0 | 0.0 | 0.0 | 0.0 | 0.0 | 0.0 | 0.0 | 0.0 |
| Chlorophyll | 0.0 | 0.0 | 0.0 | 0.0 | 0.0 | 0.0 | 0.0 | 0.0 |
| Waxes/Paraffins | 0.0 | 0.0 | 0.0 | 0.0 | 0.0 | 0.0 | 0.0 | 0.0 |
| Total (lb/hr) | 30.0 | 2.5 | 21.3 | 6.3 | 0.0 | 4.9 | 42.6 | 12.5 |
| Temp (° F.) | 125.0 | 320.0-470.0 | 150.0 | 410.0-460.0 | 150.0 | 150.0 | 150.0 | 150.0 |
| Pressure (psig) | 15.0 | −13.0 | 30.0 | 30.0 | −14.688 | 5.0 | 5.0 | 5.0 |

Table II below is exemplary of process conditions throughout a continuous vacuum fractionation system 100 in accordance with the present invention utilizing a “poor” quality raw cannabis extract as a feedstock to the system 100. In general, a “poor” ˜ quality raw cannabis extract is generally defined as a raw cannabis extract containing the following in addition to the primary separation components and/or compounds desired: amounts of the extraction solvent used; amounts of waxes and/or paraffins; amounts of light terpenes; amounts of intermediate and heavy terpenes; and, amounts of various other undefined, non-essential oil components.

When processing a “poor” quality raw cannabis extract through the present continuous vacuum fractionation system 100, a first continuous fraction unit 300 and a second continuous fraction unit 400 may be operated in a series configuration, such as is represented in the exemplary process conditions presented in Table II.

TABLE II
Exemplary Processing of a “Poor” Quality Raw Cannabis Extract
Two Continuous Fractionation Units Operating in Series

| | Stream No. | | | | | | | |
|---|---|---|---|---|---|---|---|---|
| | 1 | 2 | 3 | 4 | 5 | 6 | 7 | 8 |
| | Comp. Flow (lb/hr) | | | | | | | |
| | Ttl Raw | Frac | Frac | Frac | Frac Col #1 | Frac | System Vac | Cold |
| | Ext Feed | Col #1 | Col #1 | Col #1 | Intermed as Frac | Col #1 | Pump | Trap |
| | to System | Feed | Btms | Ovhds | Col #2 Feed | Vac Strm | Outlet | Condensate |
| CBD | 18.6 | 18.6 | 0.4 | 0.0 | 18.2 | 0.0 | 0.0 | 0.0 |
| THC | 2.7 | 2.7 | 0.1 | 0.0 | 2.6 | 0.0 | 0.0 | 0.0 |
| Lt. Terpenes | 2.3 | 2.3 | 0.0 | 0.0 | 0.0 | 2.3 | 0.0 | 2.3 |
| Inter. Terpenes | 2.3 | 2.3 | 0.0 | 0.0 | 1.1 | 1.1 | 0.0 | 1.1 |
| Hvy. Terpenes | 2.0 | 2.0 | 2.0 | 0.0 | 0.0 | 0.0 | 0.0 | 0.0 |
| Solvent | 1.2 | 1.2 | 0.0 | 0.0 | 0.0 | 1.2 | 1.2 | 0.0 |
| Heavy Oils | 0.3 | 0.3 | 0.3 | 0.0 | 0.0 | 0.0 | 0.0 | 0.0 |
| Chlorophyll | 0.2 | 0.2 | 0.2 | 0.0 | 0.0 | 0.0 | 0.0 | 0.0 |
| Waxes/Paraffins | 0.6 | 0.6 | 0.6 | 0.0 | 0.0 | 0.0 | 0.0 | 0.0 |
| Total (lb/hr) | 30.0 | 30.0 | 3.4 | 0.0 | 22.0 | 1.2 | 1.2 | 3.4 |
| Temp (° F.) | 125.0 | 125.0 | 240.0 | 150.0 | 220.0 | 85.0 | 85.0 | 25.0 |
| Pressure (psig) | 1.0 | 15.0 | −13.0 | 30.0 | 30.0 | −14.688 | −14.688 | −14.688 |
| | Stream No. | | | | | | | |
| | 9 | 10 | 11 | 12 | 13 | 14 | 15 | 16 |
| | Comp. Flow (lb/hr) | | | | | | | |
| | Direct Frac | Frac Col | Frac Col | Frac Col | Frac Col | Combined | Frac Col | Frac Col |
| | Col #2 Feed | #2 Btms | #2 Ovhds | #2 Intermed | #2 Vac Strm | Column Btms | #2 Ovhds | #2 Intermed |
| CBD | 0.0 | 0.0 | 18.2 | 0.0 | 0.0 | 0.4 | 18.2 | 0.0 |
| THC | 0.0 | 0.0 | 0.0 | 2.6 | 0.0 | 0.1 | 0.0 | 2.6 |
| Lt. Terpenes | 0.0 | 0.0 | 0.0 | 0.0 | 0.0 | 0.0 | 0.0 | 0.0 |
| Inter. Terpenes | 0.0 | 1.1 | 0.0 | 0.0 | 0.0 | 1.1 | 0.0 | 0.0 |
| Hvy. Terpenes | 0.0 | 0.0 | 0.0 | 0.0 | 0.0 | 2.0 | 0.0 | 0.0 |
| Solvent | 0.0 | 0.0 | 0.0 | 0.0 | 0.0 | 0.0 | 0.0 | 0.0 |
| Heavy Oils | 0.0 | 0.0 | 0.0 | 0.0 | 0.0 | 0.3 | 0.0 | 0.0 |
| Chlorophyll | 0.0 | 0.0 | 0.0 | 0.0 | 0.0 | 0.2 | 0.0 | 0.0 |
| Waxes/Paraffins | 0.0 | 0.0 | 0.0 | 0.0 | 0.0 | 0.6 | 0.0 | 0.0 |
| Total (lb/hr) | 0.0 | 1.2 | 18.2 | 2.6 | 0.0 | 4.6 | 18.2 | 2.6 |
| Temp (° F.) | — | 420.0-470.0 | 150.0 | 410.0-460.0 | 150.0 | 150.0 | 150.0 | 150.0 |
| Pressure (psig) | — | −13.0 | 30.0 | 30.0 | −14.688 | 5.0 | 5.0 | 5.0 |

As will be appreciated from the foregoing, the present continuous vacuum fraction system 100 provides the operational flexibility necessary to meet the demands and challenges presented by extremely variable raw cannabis extract feed stocks, as well as by the demands of ever changing markets for any of a variety of desired components and/or compounds which may be separated from raw cannabis extracts.

Turning next to the illustrative embodiment of FIG. 6, a diagrammatic representation of one illustrative embodiment of a modular fractionation column in accordance with the present invention, generally as shown as at 1000, is presented. As may be seen from FIG. 6, in at least one embodiment a modular fractionation column 1000 comprises a re-boiler 1200, which also serves as the base and support for the column 1000, a close-coupled overhead condenser 1400, and a plurality of modular fractionation stages 1600 disposed therein between. As will be appreciated by those of skill in the art, by virtue of the modular configuration of a modular fractionation column 1000, a continuous vacuum fractionation system 100 in accordance with the present invention may be quickly and easily retrofitted to accommodate additional or fewer modular fractionation stages 1600 such as may be warranted based on an assay of a raw cannabis extract to be separated via the present system 100.

A re-boiler 1200 in accordance with the present invention serves to heat at least portions of a raw cannabis extract to its boiling point to allow separation of the components and compounds of interest to take place in one or more of the plurality of modular fractionation stages 1600 of the modular fractionation column 1000. As such, the re-boiler 1200 includes one or more heater interconnects 1280 to permit interconnection to an appropriate heat source such as, by way of example only, an internal heating coil, an internal heat exchanger, an external heating jacket, etc., just to name a few. A re-boiler 1200 in at least one embodiment further comprises a re-boiler discharge 1260 through which a bottoms fraction, i.e., the heavier components or compounds in a raw cannabis extract which do not vaporize at an operating temperature and pressure of the modular fractionation column 1000, which are transferred to either a storage tank, for example, bottoms storage tank 660, or as a feed stream to a subsequent modular fractionation column 1000, such as when the system 100 is operated in a series configuration.

As further shown in FIG. 6, a re-boiler 1200 comprises a modular column interconnect 1220 to facilitate an operative interconnection between the re-boiler 1200 and the lowermost modular fractionation stage 1600 of the modular fractionation column 1000. More in particular, a modular column interconnect 1220 is configured and dimensioned to abut against and be operatively interconnected to a corresponding modular column interconnect 1620 affixed to the bottom of the lowermost modular fractionation stage 1600. In at least one embodiment, modular column interconnects 1220, 1620 comprise standard stainless steel pipe flanges corresponding to a particular pipe or column diameter. As such, in at least one embodiment of a modular fractionation column 1000 in accordance with the present invention, a re-boiler 1200 having a particular pipe or column diameter may be operatively interconnected to any of a plurality of modular fractionation stages 1600 having the same pipe or column diameter via corresponding ones of modular column interconnects 1220, 1620.

Looking next to FIG. 7, a diagrammatic representation of one illustrative embodiment of a close-coupled overhead condenser 1400 in accordance with the present invention is shown. As may be seen from FIG. 7, in at least one embodiment, a close-coupled overhead condenser 1400 comprises a modular column interface 1420 which, similar to modular column interconnects 1220, 1620, comprises a standard stainless steel pipe flange corresponding to a particular pipe or column diameter. As such, the modular column interconnect 1420 permits a close-coupled overhead condenser 1400 having a particular pipe or column diameter to abut against and be operatively interconnected to a modular column interconnect 1620 of the uppermost modular fractionation stage 1600 of a modular fractionation column 1000, in accordance with the present invention. In at least one embodiment, a close-coupled overhead condenser 1400 comprises an access port 1410 having a removable cover thereon which may be removed as needed for service and/or maintenance of the condenser 1480 disposed therein.

A close-coupled overhead condenser 1400 in accordance with at least one embodiment of the present invention comprises at least one impingement plate 1440 disposed in overlying relation to a condenser inlet 1430 at the interface between the close-coupled overhead condenser 1400 and the uppermost modular fractionation stage 1600 of a modular fractionation column 1000. As may be seen from FIG. 7, the close-coupled overhead condenser 1400 comprises a single oversized impingement plate 1440 disposed is substantially overlying relation to the condenser inlet 1430. As further shown in FIG. 7, the oversized impingement plate 1440 is disposed at an angle of approximately 45 degrees relative to the condenser inlet 1430, and further, the oversized impingement plate 1440 is operatively positioned between the condenser inlet 1430 and the condenser 1480. As will be appreciated, at least one embodiment of the present invention, an impingement plate 1440 is dimensioned and positioned so as to minimize non-vapor components from entering and contacting the condenser 1480 disposed within the close-coupled overhead condenser 1400. More in particular, an oversized impingement plate 1440 is positioned in an overlying relation to a condenser inlet 1430 so as to force all of the overhead vapors exiting the uppermost fractionation stage 1600 into contact with the impingement plate 1440 whereon non-vaporized components will coalesce and drop back into the uppermost fractionation stage 1600 for further processing therein. This is particularly important when processing “poor” quality raw cannabis extract as there is a greater likelihood of unwanted non-vapor components, such as extraction solvent(s) and/or other extraction contaminants, reaching the overhead condenser 1400 when a “poor” quality cannabis extract is being processed.

With reference once again to the illustrative embodiment of FIG. 7, a close-coupled overhead condenser 1400 in accordance with the present invention further comprises a condenser 1480 which is positioned in the close-coupled overhead condenser 1400 downstream of the impingement plate 1440 and is configured and disposed to contact the vapor components and/or compounds in the overhead fraction exiting the uppermost modular fractionation stage 1600 of a modular fractionation column 1000. The condenser 1480 includes condenser interconnects 1490 through which an appropriate cooling fluid is permitted to flow into and through the condenser 1480. As will be appreciated, the heated vapor components and/or compounds in the overhead fraction will cool and condense into a liquid state upon contact with the condenser 1480 and the close-coupled overhead condenser 1400 of the present invention. An overhead discharge 1460 is provided on a lower portion of the close-coupled overhead condenser 1400 to permit the transfer the overhead fraction which has been cooled and condensed into a liquid state in the close-coupled overhead condenser 1400.

Finally, FIG. 8 presents a diagrammatic representation of one illustrative embodiment of a modular fractionation stage 1600 in accordance with the present invention. As shown in FIG. 8, the modular fractionation stage 1600 comprises modular column interconnects 1620 disposed at opposite ends thereof. As before, in at least one embodiment, modular column interconnects 1620 comprise standard stainless steel pipe flanges corresponding to a particular pipe or column diameter. As such, in at least one embodiment of a modular fractionation column 1000 in accordance with the present invention, a plurality of modular fractionation stages 1600 having a particular pipe or column diameter may be operatively interconnected to one another, as well as to a re-boiler 1200 and/or a close-coupled overhead condenser 1400 having the same pipe or column diameter via corresponding ones of modular column interconnects 1220, 1420, 1620. By virtue of standardized design of the modular fractionation stages 1600 in accordance with the present invention, economies of scale may be realized via mass production of a plurality of modular fractionation stages 1600 in various pipe or column diameters in order to accommodate processing of any variety of raw cannabis extract quality and/or required process throughputs.

Each modular fractionation stage 1600 in accordance with at least one embodiment of the present invention includes a stage feed 1640 and a stage discharge 1660. As will be appreciated, depending on the quality of the raw cannabis extract and/or the separation components and/or compounds desired to be obtained, a feed stream may be directed to one or more of a plurality of modular fractionation stages 1600 via a corresponding stage feed 1640 and/or a side stream fraction may be withdrawn from one or more of the plurality of modular fractionation stages 1600 via a corresponding stage discharge 1660. As further shown in the illustrative embodiment of FIG. 8, a modular fractionation stage 1600 in accordance with the present invention further comprises stage internals 1650. As will be appreciated by those of skill in the art, stage internals 1650 can include random or structured packing, condensate collection plates, down comers, such as are known for use in fixed fractionation columns.

Thus, it will be appreciated by those of skill in the art that the present continuous vacuum fractionation system 100 employing one or more modular fractionation column 1000, each having a close-coupled overhead condenser 1400 having an impingement plate 1440, and operable in either a series or parallel configuration in accordance with the present invention, may be utilized for the separation of numerous desired components and/or compounds, as well as undesirable solvents present in variable amounts in raw cannabis extracts prepared by any of a variety of extraction techniques, and thus having a range of qualities from “good” to “poor.” By virtue of the modular construction, the present continuous vacuum fraction system 100 may be readily modified by adding or removing modular fraction stages 1600 for a modular fractionation column 1000 as warranted based on an assay of a raw cannabis extract. Further the utilization of modular components allows economies of scale to be realized by mass production of the various modular fractionation column components, thereby making the present system 100 considerably more economic and efficient to install, operate, and maintain.

Since many modifications, variations, and changes in detail can be made to the described preferred embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

1. A continuous vacuum fractionation system for separation of one or more of a plurality of components present in a raw cannabis extract, wherein the raw cannabis extract is prepared by any of a variety of extraction techniques, said system comprising:
a first continuous fractionation unit comprising a
first modular fractionation column having a first re-boiler, a first close-coupled overhead condenser, and at least one first modular fractionation stage;
said first close-coupled overhead condenser comprising at least one oversized impingement plate disposed in an at least partially overlying relation to a first condenser inlet from said at least one first modular fractionation stage to minimize non-vapor components from entering and contacting a first condenser therein;
a first feed stream comprising an amount of the raw cannabis extract delivered to said at least one first modular fractionation stage of said first continuous modular fractionation column;
said first continuous modular fractionation column dimensioned and configured to separate the first feed stream into at least a first overhead fraction, a first bottoms fraction, and a first side stream fraction;
a second continuous fractionation unit comprising a second modular fractionation column disposed in fluid communication with at least said first continuous modular fractionation unit;
said second modular fractionation column having a second re-boiler, a second close-coupled overhead condenser, and at least one second modular fractionation stage;
said second close-coupled overhead condenser comprising at least one oversized impingement plate disposed in an at least partially overlying relation to a second condenser inlet from said at least one second modular fractionation stage to minimize non-vapor components from entering and contacting a second condenser therein;
a second feed stream delivered to said at least one second modular fractionation stage of said second continuous modular fractionation column, wherein said second feed stream comprises one or more of the raw cannabis extract, the first overhead fraction, the first bottoms fraction, and the first side stream fraction;
said second continuous modular fractionation column configured to separate the second feed stream into at least a second overhead fraction, a second bottoms fraction and a second side stream fraction; and
a vacuum assembly disposed in communication with at least said first continuous fractionation unit and said second continuous fractionation unit, said overhead vacuum assembly maintaining at least said first modular fractionation column and said second modular fractionation column under a predetermined vacuum during operation.

2. The continuous vacuum fractionation system as recited in claim 1 wherein said first modular fractionation column comprises a plurality of first modular fractionation stages, each of said plurality of first modular fractionation stages interconnected in a series arrangement between said first re-boiler and said first close-coupled overhead condenser.

3. The continuous vacuum fractionation system as recited in claim 2 wherein a number of said plurality of first modular fractionation stages is selected based in part upon an assay of the raw cannabis extract being processed.

4. The continuous vacuum fractionation system as recited in claim 1 wherein said second modular fractionation column comprises a plurality of second modular fractionation stages, each of said plurality of second modular fractionation stages interconnected in a series arrangement between said second re-boiler and said second close-coupled overhead condenser.

5. The continuous vacuum fractionation system as recited in claim 4 wherein a number of said plurality of second modular fractionation stages is selected based in part upon an assay of the raw cannabis extract being processed.

6. The continuous vacuum fractionation system as recited in claim 1 wherein said first continuous fractionation unit comprises a first re-boiler pump disposed to transfer the first bottoms fraction from said first re-boiler of said first modular fractionation column, and a first re-boiler airlock disposed between said first re-boiler and said first re-boiler pump configured to maintain sufficient pressure head at a suction side of said first re-boiler pump.

7. The continuous vacuum fractionation system as recited in claim 1 wherein said first continuous fractionation unit comprises a first side stream pump disposed to transfer the first side stream fraction from said at least one first modular fractionation stage of said first modular fractionation column, and a first side stream airlock disposed between said at least one first modular fractionation stage and said first side stream pump configured to maintain sufficient pressure head at a suction side of said first side stream pump.

8. The continuous vacuum fractionation system as recited in claim 1 wherein said second continuous fractionation unit comprises a second re-boiler pump disposed to transfer the second bottoms fraction from said second re-boiler of said second modular fractionation column, and a second re-boiler airlock disposed between said second re-boiler and said second re-boiler pump configured to maintain sufficient pressure head at a suction side of said second re-boiler pump.

9. The continuous vacuum fractionation system as recited in claim 1 wherein said second continuous fractionation unit comprises a second side stream pump disposed to transfer the second side stream fraction from said at least one second modular fractionation stage of said second modular fractionation column, and a second side stream pump airlock disposed between said at least one second modular fractionation stage and said second side stream pump configured to maintain sufficient pressure head at a suction side of said second side stream pump.

10. The continuous vacuum fractionation system as recited in claim 1 wherein said vacuum assembly comprises at least one vacuum pump disposed in communication with said first continuous fractionation unit and said second continuous fractionation unit via a vacuum line, said at least one vacuum pump is dimensioned and configured to maintain at least said first modular fractionation column and said second modular fractionation column under a predetermined vacuum during operation.

11. The continuous vacuum fractionation system as recited in claim 10 wherein said predetermined vacuum is about 0.1 millimeter of mercury to about 100 millimeters of mercury.

12. The continuous vacuum fractionation system as recited in claim 10 wherein said predetermined vacuum is about 0.7 millimeter of mercury.

13. The continuous vacuum fractionation system as recited in claim 10 wherein said predetermined vacuum is about 50 millimeters of mercury.

14. The continuous vacuum fractionation system as recited in claim 10 wherein said first continuous fractionation unit comprises a first reflux tank wherein the first overhead fraction is separated into a first overhead liquid reflux which is discharged via a first reflux pump, and a first overhead vapor which is discharged into said vacuum line.

15. The continuous vacuum fractionation system as recited in claim 10 wherein said second continuous fractionation unit comprises a second reflux tank wherein the second overhead fraction is separated into a second liquid reflux which is discharged via a second reflux pump, and a second reflux vapor which is discharged into said vacuum line.

16. The continuous vacuum fractionation system as recited in claim 10 wherein said vacuum assembly comprises a cold trap disposed in said vacuum line prior to said at least one vacuum pump, said cold trap condenses light extraction contaminants present in a first reflux vapor and a second reflux vapor in said vacuum line to minimize the amount of light extraction contaminants which enter said at least one vacuum pump.

17. The continuous vacuum fractionation system as recited in claim 16 wherein said vacuum assembly further comprises a chiller disposed in communication with said cold trap, said chiller configured to maintain said cold trap at a temperature of less than zero degrees Fahrenheit.

18. The continuous vacuum fractionation system as recited in claim 16 wherein said chiller is configured to maintain said cold trap at a temperature of about minus ten degrees Fahrenheit.

19. The continuous vacuum fractionation system as recited in claim 16 wherein said vacuum assembly further comprises a condensate discharge dimensioned and configured to transfer the condensed light extraction contaminants from said vacuum line prior to said at least one vacuum pump.

20. The continuous vacuum fractionation system as recited in claim 1 further comprising a final product storage assembly including an overhead storage tank, a side stream storage tank, and a bottoms storage tank.
